(19) United States
(12) Patent Application Publication
(10) Pub. No.: US 2019/0139636 A1
(43) Pub. Date: May 9, 2019

(54) COMPUTERIZED ASSISTANCE OF MEDICATION REGIMEN REVIEWS

(71) Applicant: Managed Health Care Associates, Inc., Florham Park, NJ (US)

(72) Inventors: Arunraj Chandrasekaran, Lake Hiawatha, NJ (US); Stewart Bennet Davis, Somerset, NJ (US); Joseph M. Loeper, Portland, OR (US)

(21) Appl. No.: 16/172,071

(22) Filed: Oct. 26, 2018

Related U.S. Application Data
(60) Provisional application No. 62/582,066, filed on Nov. 6, 2017.

(51) Int. Cl. G16H 20/10; G16H 40/40; G16H 80/00; G16H 10/60

(52) U.S. Cl. CPC G16H 20/10; G16H 10/60; G16H 80/00; G16H 40/40

(57) ABSTRACT

A computer-implemented method for performing Medication Regimen Reviews (MRR) includes synchronizing a local medication regimen database stored on a client computing device with a remote medication regimen database stored on a server computing device. The local medication regimen database includes medication regimens for patients residing at one or more facilities. Medication regimen recommendations are created based on input received via a client interface on the client computing device. Each medication regimen recommendation is created by a recommendation process. These medication regimen recommendations may then be stored in a local medication recommendations database on the client computing device.

### CROSS REFERENCE TO RELATED APPLICATIONS

This application claims the benefit of U.S. Provisional Patent Application Ser. No. 62/582,066, filed on Nov. 6, 2017, entitled “Computerized Assistance of Medication Regimen Reviews,” the entire contents of which are hereby incorporated by reference herein.

### TECHNICAL FIELD

The present disclosure relates generally to techniques for automating or semi-automating the process of performing a Medication Regimen Review (MRR) using a plurality of computing devices. The techniques described herein may be applied for performing MRRs, for example, in Long Term Care (LTC) facilities.

### BACKGROUND

A consultant pharmacist is a pharmacist that provides advice on the use of medications and pharmacy services to individuals, referred to herein as “patients.” This advice may be provided, for example, to the patient's physician, nursing staff, or the medical facility where the patient is a resident. The consultant pharmacist advice is formulated by performing a Medication Regimen Review (MRR). During the MRR, the consultant pharmacist identifies actual or potential drug therapy problems and makes recommendations for resolving those problems. The process of performing MRRs is an ever-increasing complex task, since there are a myriad of federal regulations surrounding appropriate use of medications.

Conventional techniques for performing MRRs often rely entirely on manual input, either via “pen and paper” or using an existing word processor, spreadsheet, or database program for data entry/printing of reports. However, performing MRRs manually is not sustainable as a long-term solution for a pharmacist to effectively serve as consultant pharmacist for the facility. Manual data entry can be error prone due to humans entering the wrong information or information which is irrelevant to the MRR. Moreover, because all data needs to be manually entered, the scale of the MRR is limited by its data entry requirements.

### SUMMARY

Embodiments of the present invention address and overcome one or more of the above shortcomings and drawbacks, by providing methods, systems, and apparatuses related to automating or semi-automating the process of performing MRRs using a plurality of computing devices.

According to some embodiments, a computer-implemented method for performing a Medication Regimen Review (MRR) includes synchronizing a local medication regimen database stored on a client computing device with a remote medication regimen database stored on a server computing device. In one embodiment, synchronization of the local medication regimen database is performed automatically in response to the client computing device receiving an alert message from the server computing device. The local medication regimen database includes medication regimens for patients residing (i.e., resident) at one or more facilities. Medication regimen recommendations are created based on input received via a client interface on the client computing device. Each medication regimen recommendation is created by a recommendation process that includes receiving a selection of a patient via the client interface and retrieving a medication regimen corresponding to the patient from the local medication regimen database. During the recommendation process, recommendation phrasing text is retrieved from a local recommendation phrasings database stored on the client computing device. The recommendation phrasing text is automatically retrieved from the local recommendation phrasings database in response to a macro executed on the client computing device via the client interface. A description of one or more changes to the medication regimen is generated, along with a recommendation classification describing an MRR activity based on the recommendation phrasing text. The medication regimen recommendation is created based on the description and the recommendation classification. The medication regimen recommendations may then be stored in a local medication recommendations database on the client computing device.

Some embodiments of the aforementioned method also include synchronizing a local prescription drug plan (PDP) database stored on the client computing device with a remote PDP database stored on the server computing device and identifying a patient PDP based on the identification of a particular patient. The PDP database comprises a listing of medication coverage for one or more PDPs. A customized formulary is created from the local PDP database comprising a listing of medication and coverage information for each medication corresponding to the patient PDP. In response to receiving user selection of a specific medication in the customized formulary, one or more therapeutic alternatives to the specific medication are automatically displayed. In response to this display, a selection of a specific therapeutic alternative to the specific medication may then be received. The specific therapeutic alternative to the specific medication may then be used to generate the description of one or more changes to the medication regimen.

In some embodiments of the methods discussed above, a local clinical monograph database stored on the client computing device is synchronized with a remote clinical monograph database stored on the server computing device. The local clinical monograph database comprises clinical monographs corresponding to medications in the local medication regimen database. Next, in response to receiving a request to display a clinical monograph corresponding to a specific medication in a particular patient's medication regimen, a specific clinical monograph may be retrieved from the local clinical monograph database and the specific clinical monograph can be displayed in the client interface.

In some embodiments of the aforementioned methods, a local medication interactions database stored on the client computing device is synchronized with a remote medication interactions database stored on the server computing device. The local medication interactions database comprises descriptions of medication interactions corresponding to medications in the local medication regimen database. Specific medication interactions corresponding to a particular patient's medication regimen may be retrieved from the local medication interaction database and the specific medication interactions may be displayed in the client interface.

In some embodiments of the methods discussed above, an outcome record is received via input into the client interface. This outcome record comprises a description of an outcome resulting from performance of a specific medication regimen recommendation. The outcome record may then be stored on the client computing device in a local outcome record database. In one embodiment, the outcome record further comprises (i) a description of an outcome resulting from performance of the specific medication regimen recommendation, (ii) a first number specifying a number of adverse drug interactions identified after performing the specific medication regimen recommendation, and (iii) a second number specifying a number of adverse drug interactions avoided after performing the specific medication regimen recommendation. Additionally (or alternatively), the outcome record may include a description of a medication order change which occurred as a result of performance of the specific medication regimen recommendation. This description of the medication order change may include, for example, psychoactive medication classification information related to the medication order changes. The local outcome record database and the local medication recommendations database may be synchronized with corresponding database on the server computing device.

In some embodiments, reports may be generated for a particular facility by receiving an identification of a facility and a report generation request via the client interface. Based on the facility, a list of patients residing in the facility may be identified. Next, medication recommendations corresponding to patients may be retrieved from the local medication recommendations database or the remote medication recommendations database. Additionally, outcome records corresponding to the patients may be retrieved from one of the databases. Reports may then be generated based on these medication recommendations and outcome records.

In some embodiments, time tracking functionality may be incorporated into the methods discussed above. For example, in one embodiment, the time spent by a user in performing the recommendation process is tracked. The reports generated during the method may include at least one report indicating the time spent.

According to another aspect of the present invention, a computer-implemented method for performing MRRs includes creating medication regimen recommendations based on input received via a web-based client interface on a client computing device. Each medication regimen recommendation is created by a recommendation process that includes receiving a selection of a patient via the web-based client interface and retrieving a medication regimen corresponding to the patient from a medication regimen database stored on a server computing device. Recommendation phrasing text is retrieved from a recommendation phrasings database stored on the server computing device in response to receipt of a phrasing request via the web-based client interface. A description of one or more changes to the medication regimen is generated, along with a recommendation classification describing an MRR activity based on the recommendation phrasing text. The medication regimen recommendation is created based on the description and the recommendation classification. Then, the medication regimen recommendations are stored in a medication recommendations database on the server computing device.

According to other embodiments of the present invention, a system for performing MRRs on a client computing device comprises local database, a report upload queue, and a client application. The local databases comprise a medication recommendations database and an outcome record database. The report upload queue is synchronized to an MRR reports database stored on a server computing device. The client application is configured to (i) generate one or more MRR reports using medication recommendations database and the outcome record database, and (ii) enter each MRR report into the report upload queue.

Additional features and advantages of the invention will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawings embodiments that are presently preferred, it being understood, however, that the invention is not limited to the specific instrumentalities disclosed. Included in the drawings are the following Figures:

FIG. 1 shows a conceptual review of a system for performing MRRs in a networked computing environment, according to some embodiments.

FIG. 2 illustrates a method for performing MRRs, according to some embodiments;

FIG. 3 provides an illustration of the process for uploading data between the Client Application and the Server Application, according to some embodiments;

FIG. 4 provides another example implementation of the upload and download architecture, as it may be implemented in some embodiments;

FIG. 5A provides an example management console for accessing worksheets, according to some embodiments;

FIG. 5B shows a GUI for worksheet creation, according to some embodiments;

FIG. 5C shows an example interface for accessing worksheets, according to some embodiments;

FIG. 5D shows and additional example interface for accessing worksheets, according to some embodiments;

FIG. 5E provides an example of a computed worksheet; and

FIG. 6 illustrates an exemplary computing environment within which embodiments of the invention may be implemented.

### DETAILED DESCRIPTION

Embodiments of the present invention address and overcome one or more of the above shortcomings and drawbacks, by providing methods, systems, and apparatuses related to an application intended for use by consultant pharmacists performing Medication Regimen Reviews (MRRs) in Long Term Care (LTC) facilities and other similar facilities where patient medications are periodically reviewed. The application discussed herein facilitates tasks such as importing of pharmacy dispensing data, screening of imported medication orders as they relate to current regulations, and the creation of MRR recommendations. Based on information collected via the application, the pharmacist can perform analytics and generate reports providing a detailed view of the pharmacist's recommendations. These reports can then be shared, for example, for billing purposes.

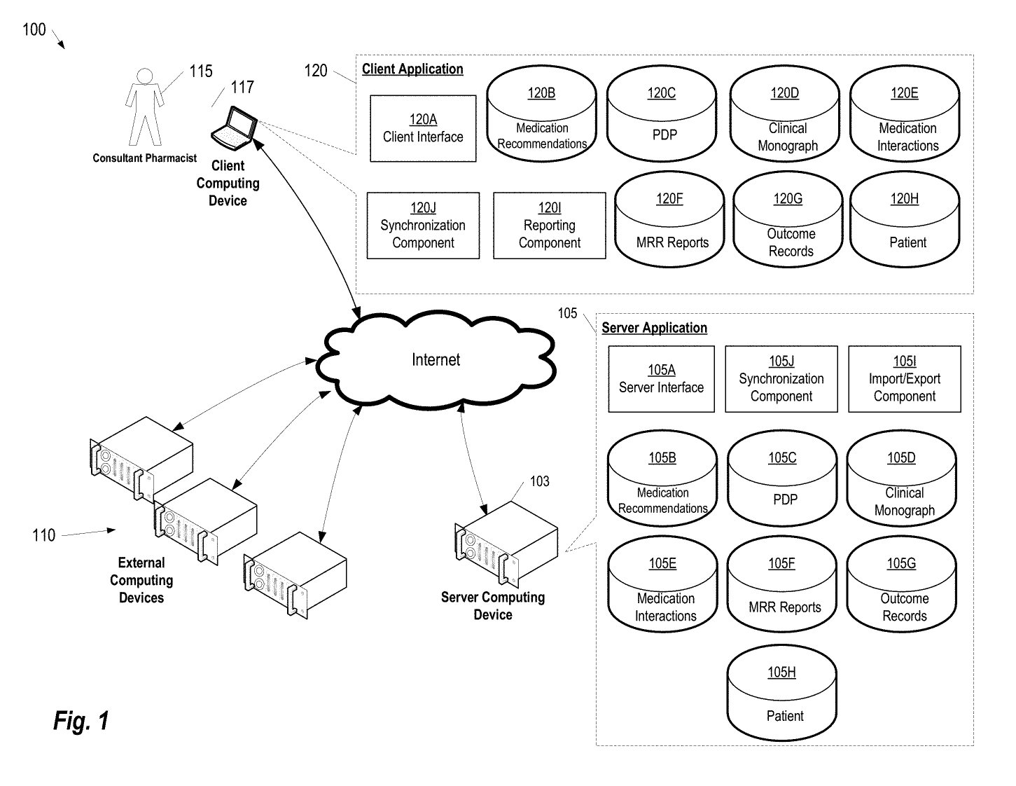

FIG. 1 shows a conceptual review of a system 100 for performing MRRs in a networked computing environment, according to some embodiments. Briefly, a Consultant Pharmacist 115 (or other user) operates a Client Computing Device 117 which executes a Client Application 120. The Client Computing Device 117 can be, for example, a desktop computer or a tablet computing device. The Client Application 120 communicates with a Server Application 105 hosted on a Server Computing Device 103 (e.g., an application server computer). In the example of FIG. 1, communication between the Client Application 120 and the Server Application 105 takes place over the Internet; however, it should be understood that any network known in the art may be used for network communications. Thus, for example, an intranet can be used in other embodiments of the present invention. The Server Computing Device 103 communicates with one or more External Computing Devices 110 to collect data which is relevant to performing MRRs.

The Client Application 120 includes a Client Interface 120A that provides a graphical user interface (GUI) through which the Consultant Pharmacist 115 can perform MRR activities. For example, the Client Interface 120A may be used to view patient medical regimens, make recommendations of changes to those medical regimens, and document outcomes resulting from the recommendations. Additionally, the Consultant Pharmacist 115 can use the Client Interface 120A to utilize with a Reporting Component 1201 within the Consultant Pharmacist 115. The Reporting Component 1201 is used to generate reports regarding medical regimen recommendations, outcomes, etc. The Reporting Component 1201 may customize each report to the Consultant Pharmacist 115 by including features such as user-defined report title, custom header/footer, a company logo, or the scanned digital signature of the Consultant Pharmacist 115.

The GUI of the Client Application 120 is intended to provide a flexible work environment allowing for customization of displays of data grids and user interfaces to improve the user experience. Thus, in some embodiments, the Client Interface 120A may have one or more customization components (not shown in FIG. 1) that allow users to modify various aspects of how the Client Interface 120A is presented. For example, through the customization components, the colors, sounds, and/or the fonts used may modified. In some embodiments, the visualization components are used to provide localization of the Client Interface 120A. For example, the visualization components may display certain logos for a group of users or display text in a certain language.

The Client Application 120 is designed to operate in an online or offline manner. Thus, the Client Application 120 can be utilized even if the Client Computing Device 117 is not connected to the Internet or the Server Computing Device 103 or is otherwise not available for communication. To support offline MRR functionality, the Client Application 120 includes local databases 120B-120H which store MRR-relevant information. In the example of FIG. 1, there are seven local databases shown: a Medication Recommendations Database 120B, a Prescription Drug Plan (PDP) Database 120C, a Clinical Monograph Database 120D, a Medication Interactions Database 120E, a MRR Reports Database 120F, an Outcome Records Database 120G, and a Patient Database 120H. Each of these databases is described in further detail below. It should be noted that, although FIG. 1 only shows six databases, additional databases of MRR relevant information may be included in other embodiments of the present invention. It should also be noted that the illustration of multiple databases in FIG. 1 is for conceptual purposes and should not be understood to be an architectural constraint. For example, multiple databases can be combined, possibly in a single database, to provide the same functionality as the various databases shown in FIG. 1.

The databases described herein may be implemented using any technique known in the art. For example, in some embodiments, a SQL-based database such as Microsoft SQL Server may be used. In other embodiments, a No-SQL database with a table equivalent structure may be employed. As is understood in the art, the term “No-SQL” is used to define a class of data stores that are non-relational in their design. There are various types of No-SQL databases which may be generally grouped according to their underlying data model. These groupings may include databases that use column-based data models (e.g., Cassandra), document-based data models (e.g., MongoDB), key-value based data models (e.g., Redis), and/or graph-based data models (e.g., Allego). Any type of No-SQL database may be used to implement the various embodiments described herein. For example, in one embodiment, MongoDB software is used to provide the underlying functionality of the database.

Continuing with reference to FIG. 1, the Patient Database 120H stores patient information for one or more facilities. The term “patient” is used herein to denote any individual that is a resident of the facility or is otherwise receiving treatment through a facility. The information included in the Patient Database 120H may include, for example, patient identifying information, patient demographic information, patient PDP information, and each patient's current medication regimen (including medication orders, labs, etc.).

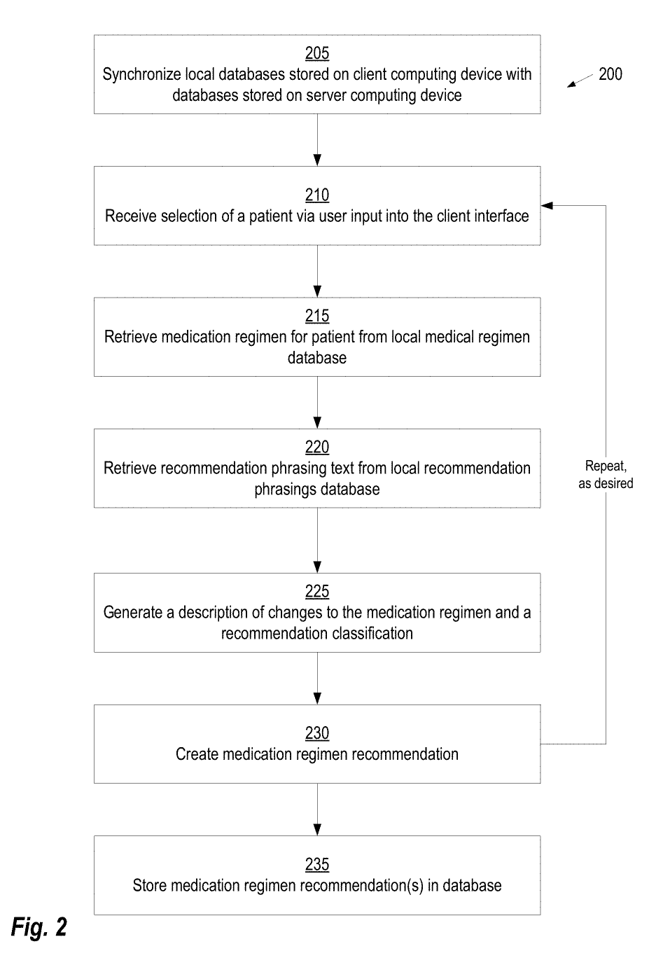

The Medication Recommendations Database 120B stores recommendations for medication regimen changes made by the Consultant Pharmacist 115 via the Client Interface 120A. Recommendations are described in further detail below with respect to FIG. 2. The information stored in the Medication Recommendations Database 120B includes text describing each recommendation. Additional fields may also be specified for each recommendation. For example, in some embodiments, the Consultant Pharmacist 115 can specify the priority of a recommendation (e.g., high, normal, or low), category information (e.g., “medication reduction request”) and information characterizing the MRR activity performed when the recommendation is generated (e.g., “MRR Admit”). The Medication Recommendations Database 120B may additionally store routing instructions indicating where each recommendation should be routed to for further processing (e.g., “Nursing Staff”). The Outcome Records Database 120G stores outcome records generated by the Consultant Pharmacist 115 via the Client Interface 120A. Each outcome record provides a description of the outcome of a particular recommendation. Additionally, the outcome record may characterize the outcome (e.g., positive, negative, etc.) and indicate whether any adverse drug reactions were identified or avoided as a result of the recommendation.

PDPs such as Medicare have a list of covered drugs, also known as a “formulary.” The formulary is generally quite extensive and can be updated frequently; thus one challenging task for the Consultant Pharmacist 115 is to keep well-informed and up-to-date on which drugs are and are not included in a formulary. To aid the Consultant Pharmacist 115 in making recommendations, the PDP Database 120C stores formulary information regarding a plurality of PDPs. This information may specify, for example, a listing of covered individuals, benefits, exclusions, co-pay rules, etc. For example, the Consultant Pharmacist 115 may use information from the PDP Database 120C to identify a particular resident's PDP and then be able to verify prescription drug coverage based upon that actual PDP. Additionally, PDP Database 120C may be used to identify therapeutic alternatives to particular medications in a patient's medication regimen that would be covered under a given PDP.

The Clinical Monograph Database 120D stores a plurality of drug monographs. Each monograph specifies the kinds and amounts of ingredients a drug or group of drugs may contain. The monograph may also include directions for use of the drug, conditions under which the drug should be administered, and any contraindications. The Client Interface 120A retrieves information from the Clinical Monograph Database 120D, for example, based on medication information presented in imported pharmacy dispensing data or manually entered data.

The Medication Interactions Database 120E stores information indicating interactions between various medications that may be included in patient regimens. In some embodiments, the medication interactions may be presented to the Consultant Pharmacist 115 in the Client Interface 120A to guide the Consultant Pharmacist 115 in making recommendations. In other embodiments, functionality in the Client Interface 120A may automatically query the Medication Interactions Database 120E as a recommendation is generated. For example, the Medication Interactions Database 120E may be searched based on keyword. If the Consultant Pharmacist 115 enters a new medication into a recommendation for a patient, the interactions for the other medications in the patient's medication regimen can be automatically parsed to determine whether any interactions need to be flagged for the Consultant Pharmacist 115. If an interaction exists, the Consultant Pharmacist 115 may be alerted, for example, by changing the text color of the new medicine in the recommendation description or displaying another visual notification in the Client Interface 120A.

The MRR Reports Database 120F stores reports generated by the Reporting Component 1201. The Reporting Component 1201 may store reports in various formats generally known in the art including, without limitation, JSON (JavaScript Object Notation), Microsoft Word Document (.doc), a Microsoft Excel Spreadsheet (.xls), or the Portable Document Format (PDF). In this way, reports can be shared outside of the Client Application 120, for example via secure email leveraging the DIRECT healthcare standards, between the Consultant Pharmacist 115 and the staff of an LTC facility or other patient facility.

In some embodiments, a time tracker component (not shown in FIG. 1) may be added to the Client Application 120 to allow the Consultant Pharmacist 115 to document and qualify time spent on various consulting activities. The tracked information may be used, for example, to assist with billing and to assist the facility in tracking time as part of the payroll-based journal requirements. Various features generally known in the art of time tracking may be incorporated into the time tracker module. For example, the time tracker module may include features such as manual or automated time entry, Global Positioning System (GPS) location tracking customizable alerts, and reminders, report generation, and/or vacation tracking.

The Server Computing Device 103 includes databases 105B-105H which store the same information as is stored in the databases 120B-120H on the Client Computing Device 117. For clarity, the databases 105B-105H on the Server Computing Device 103 are referred to herein as “remote databases,” while the databases 120B-120H on the Client Computing Device 117 are referred to herein as “local databases.” A Synchronization Component 120J on the Client Computing Device 117 communicates with a Synchronization Component 105J on the Server Computing Device 103 to synchronize data between the local and remote databases. Synchronization methodologies are generally known in the art and, in general, any technique for synchronization may be utilized by the Synchronization Components 105J, 120J. In some embodiments, synchronization is triggered by an action by Consultant Pharmacist 115 via the Client Interface 120A. For example, the Consultant Pharmacist 115 may click a button requesting synchronization, synchronization may be performed during Client Application 120 startup or shutdown, or synchronization may be performed periodically. Each individual database may be synchronized individually. For example, the Medication Interactions Databases 120E, 105E may only need to be synchronized monthly, while the Medication Recommendation Databases 120B may be synchronized continuously if the Client Computing Device 117 has connectivity to the Server Computing Device 103. In some embodiments, the Server Application 105 may push updates to the local databases as they become available, thus removing the need for the Client Application 120 to check for updates. The syncing functionality can be designed to provide features such as end-to-end encryption, organization of data into queues to prioritize certain uploads, MD5 checksum checks or file comparisons to ensure correct copying, and automatic reconnection if the client and server lose their connection during uploading.

Aside from synchronizing with the Client Application 120, the Synchronization Component 105J of the Server Application 105 may also be used to retrieve data from one or more External Computing Devices 110 storing data relevant for MRRs. For example, one of the External Computing Devices 110 may include a database of medication interactions. The Synchronization Component 105J may synchronize this external database with the Medication Interactions Database 105E in the Server Application 105. Any new information will then be sent to the Medication Interactions Database 120E on the Client Application 120 when it synchronizes with the Server Application 105.

The Import/Export Component 1051 included in the Server Application 105 allows for multiple data exchange opportunities with other consultant pharmacists using the Client Application 120 or other client applications. This functionality may be applied, for example, to support “team consulting,” where two or more consultant pharmacists perform MRRs at the same facility, vacation coverage, and overall quality assurances. Various MRR, reference, statistical study, and worksheet reports may be generated using the Import/Export Component 1051. The Import/Export Component 1051 may export data into various formats generally known in the art including, without limitation, JSON (JavaScript Object Notation), Health Level 7 Fast Healthcare Interoperability Resources (HL7 FHIR), Electronic Data Interchange (EDI), Microsoft Word Document (.doc), a Microsoft Excel Spreadsheet (.xls), or the Portable Document Format (PDF). In some embodiments, the Import/Export Component 1051 also generates aggregate reports, using the same statistical data sets as used for the other reports described above. These aggregate reports allow the pharmacist to compare and contrast recommendation outcomes and medication utilization data across multiple facilities. For example, the aggregate reports may be used for larger LTC organizations that are serviced by the same consultant pharmacist.

The Server Computing Device 103 also includes a Server Interface 105A which may be used to directly access data stored in the Server Application 105. This Server Interface 105A may be used, for example, by a user wishing to view recommendation and outcome data generated by a group of consultant pharmacists in a particular organization or a group of consultant pharmacists servicing a particular facility. The Server Interface 105A may provide a GUI that allows the user to interface with the various remote databases stored on the Server Computing Device 103. In this way, the Server Interface 105A may function as a web management console. Alternatively (or additionally), the Server Interface 105A may include an Application Programming Interface (API) that allows external application to communicate with the Server Application 105. For example, in one embodiment, the Server Interface 105A implements a service-based interface using representational state transfer (REST) or a similar architectural style.

FIG. 2 illustrates a method 200 for performing MRRs, according to some embodiments. This method is intended to be performed by a client application executing on a client computing device (e.g., desktop computer, tablet, etc.) in conjunction with a server computing device. As an initial optional step, at step 205 one or more local databases stored on the client computing device may be synchronized with corresponding databases stored on the server computing device. For example, as shown in FIG. 1, the local client application may include a local medication regimen database which stores local medication regimen database includes medication regimens for patients that are residents at one or more facilities. The corresponding database for the local medication regimen database would be a remote local medication regimen database stored on the server computing device.

Continuing with reference to FIG. 2, at steps 210-230 a recommendation process is performed on the client computer device to create a plurality of medication regimen recommendations. The term “creating,” as used with reference to FIG. 2 refers to collecting, retrieving, or generating the information necessary to create a medication regimen recommendation. The recommendation process begins at step 210 where a selection of a patient is received via user input to a client interface included within the client application. For example, in some embodiments, a patient may be identified by his or her name or an alphanumeric identifier. In some embodiments, search functionality is provided to identify matching patients stored in a local database of available patients. In other embodiments, a listing of patients is provided and the user input comprises a selection of one or more of the patients in the listing. For example, patient records may be presented in a spreadsheet-type interface and individual patients may be selected by double clicking on a patient's record.

Once the patient has been selected, at step 215 a medication regimen corresponding to the selected patient is retrieved from the local medication regimen database. For example, each patient may be given a unique identifier. The key for the database may be patient identifiers; thus, the relevant information on a particular patient can be quickly accessed by extracting information pertaining to the patient's identifier.

Optionally, at step 220, recommendation phrasing text is retrieved from a local recommendation phrasings database stored on the client computing device. The recommendation phrasing text includes text snippets which may be included directly into recommendations, thus accelerating the recommendation practice. In some embodiments, the user has the ability to customize the local recommendation phrasings database for the user's own practice and to share with colleagues. The recommendation phrasing text may include, for example, pre-phrased recommendation templates generated for regulatory compliance. These templates may be populated by the user via the client interface, for example, by typing in relevant information into different fields of the template. Alternatively, different fields of the templates can be prepopulated based on recent activity performed through the client interface. For example, in some embodiments, the user can select a particular medication and a therapeutic alternative. Then, recommendation for substantiating the alternative into the patient's regimen can be automatically generated by inserting the original medication and the therapeutic alternative directly into the template.

In some embodiments, the recommendation phrasing text is automatically retrieved from the local recommendation phrasings database in response to a macro executed on the client computing device via the client interface. As is generally known in the art, a macro is an instruction that, when, executed, causes a series of additional instructions to be performed. In the context of the method 200 shown in FIG. 2, the process of generating recommendations can be standardized and a macro can be created based on the collective steps involved in the process. For example, a user may wish to create a certain type of report with certain relevant information extracted from the patient's profile. These steps can be collected and saved under a name that can later be referenced as needed. In some embodiments, macros can be chained together to provide even more powerful customization of the reports.

At step 225, the recommendation phrasing text is used to generate a description of one or more changes to the medication regimen. This may entail direct use of the recommendation phrasing text as the description of the changes or, alternatively, the recommendation phrasing text may be modified, either automatically or via the input to the client interface. In addition to the description of the changes, a recommendation classification is also generated at step 225, either automatically (e.g., based on keywords in the description) or based on input to the client interface. The recommendation classification provides an indication of the MRR activity being performed when the description is generated. Examples of possible activities that may be specified as recommendation classifications include Admit MRR (review upon admission to a facility), Interim MRR (review at a specified date), or Normal MRR (normal, periodic review). Using recommendation classifications, routine MRR activities can be delineated from more specialized activities. In turn, this can be used for targeted reporting on different clinical services. Once the description of the changes and the recommendation classification have been generated, they are used to create a medication regimen recommendation(s) at step 230. The recommendation process then continues, allowing the consultant pharmacist to create additional recommendations if desired. These additional recommendations may be for the same patient as the first recommendation, or recommendations may be created for a group of patients.

At step 235, all of the medication regimen recommendations that were created through the recommendation process are stored in a local medication recommendations database on the client computing device. Recommendations may be stored in the database as a batch, for example, after the recommendation process concludes, during periodic intervals, or when the pharmacist exits the client interface. Alternatively, step 235 can be integrated into the recommendation process, storing each recommendation as it was generated.

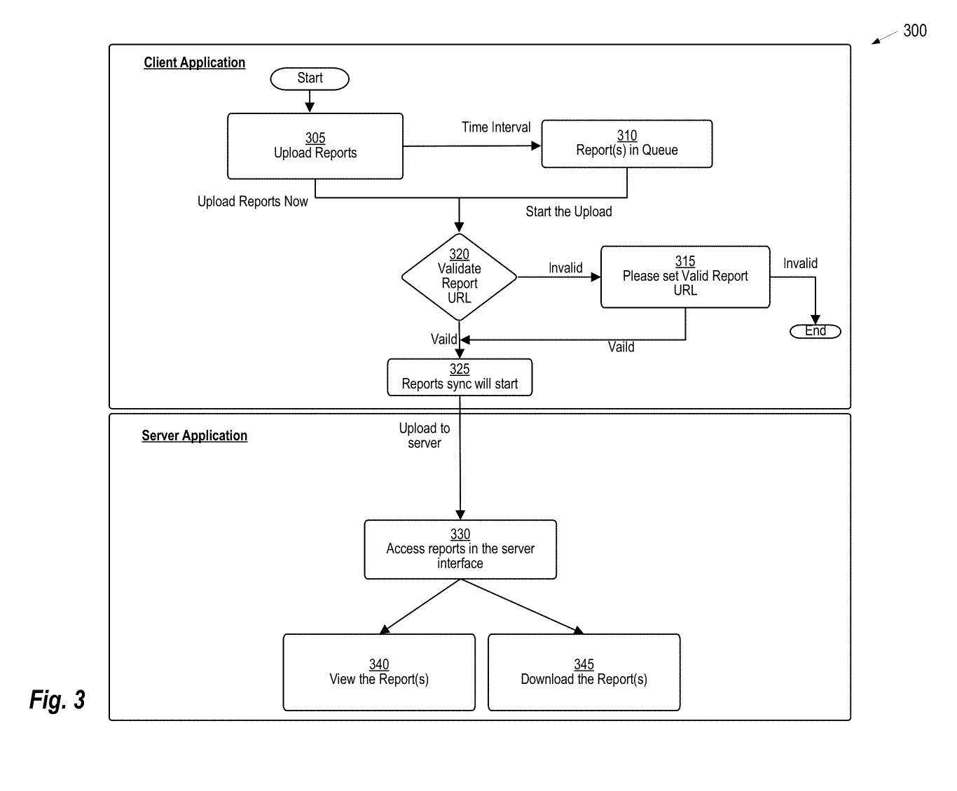

FIG. 3 provides an illustration of the process 300 for uploading data between the Client Application and the Server Application, according to some embodiments. In this example, the data being uploaded are reports detailing recommendations and/or outcomes generated using the client interface. Starting at step 305, a consultant pharmacist interacts with the client interface on a client computing device to request uploading of a report to the remote MRR reports database in the server application (see FIG. 1). This request can be specified by providing an explicit file name or, alternatively, a directory can be provided and all report files in that directory can be designated for upload.

At step 305, the consultant pharmacist 115 may be given the opportunity to specify a time interval for uploading. This time upload allows the user to batch files in queue for uploading. Also, depending on the file size of each report and the bandwidth of the client computing device's network connection, it could take a great deal of time to transfer all of the reports. In this case, a time interval may be automatically designated by the software on the client machine to delay uploading. If a time interval is specified, the report is entered into a queue at step 310.

Proceeding either directly from step 305 or from step 310, a report is uploaded by first validating its Uniform Resource Locator (URL) at step 320. In some embodiments, a single URL is used for all of the reports. This URL may be, for example, the domain of the server by itself or it may be domain with path information unique to the user. In other embodiments, each report may have a unique URL. In this case, the validation performed at step 320 could include, for example, verifying that the formatting of the URL is correct, that the report identifier is unique, and/or that the URL identifies an accessible server. Additionally, the validating step could be used to enforce certain security procedures, such as requiring each report upload to use end-to-end encryption or some other form of secure transfer. If the URL is determined to be invalid, the user is prompted at step 315 to set a valid report URL in the client device (e.g., via an options setting). If the URL continues to be invalid, the process ends. However, if the URL is valid (either following step 315 or step 320), then processing continues to step 325.

At step 325, the reports are synced between the local reports database on the client application and the corresponding remote database on the server application. For example, for a single report, transmission can be performed using an FTP PUT or HTTP POST request. For multiple reports, syncing software on the client works in conjunction with the server to transmit each item in the queue. Once in the database, users can access the reports via the server interface of the server application (see FIG. 1) at step 330. Via the server interface, a user can view the report (step 340) in a web browser. The user can also download the report (step 345) onto the user's device in the report's native format or, in some embodiments, the report may be converted to one or more standard formats such as JSON (JavaScript Object Notation), Health Level 7 Fast Healthcare Interoperability Resources (HL7 FHIR), Electronic Data Interchange (EDI), Microsoft Word Document (.doc), a Microsoft Excel Spreadsheet (.xls), or the Portable Document Format (PDF).

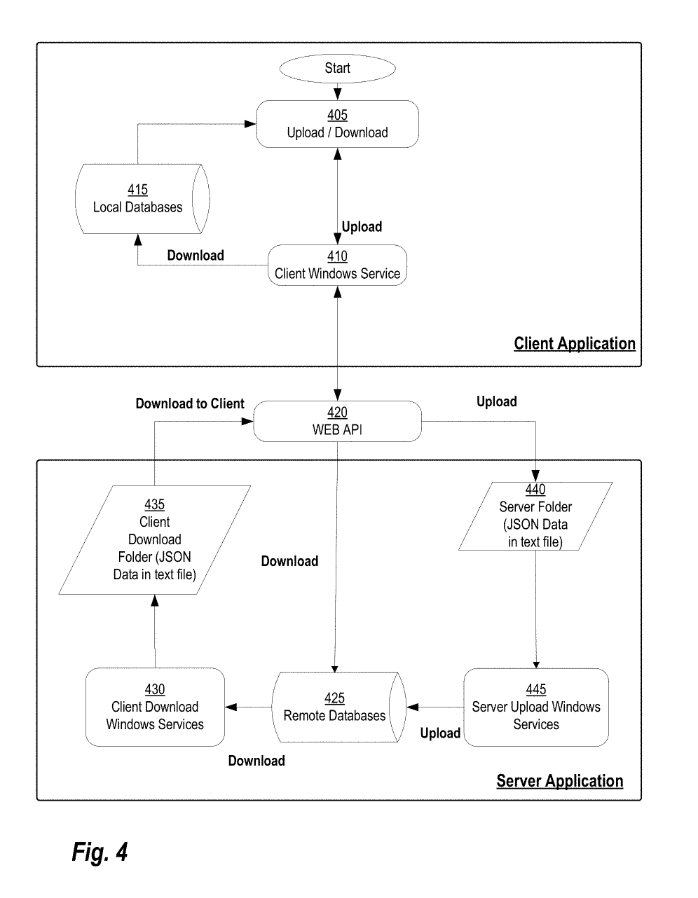

FIG. 4 provides another example implementation of the upload and download architecture, as it may be implemented in some embodiments. In this case, it is assumed that the client application is a Microsoft Windows-based application with a Client Windows Service 410 which facilitates uploading and downloading. As is generally understood in the art, a Windows service is a computer program that operates in the background. The Client Windows Service 410 may be configured to start when the operating system of the client device is first started or, alternatively, the Client Windows Service 410 may be started as needed. Once the Client Windows Service 410 is running, the user can provide an upload or download request to the Client Windows Service 410 (as shown in step 405). For downloads, the Client Windows Service 410 retrieves the requested file from the server via a Web-based API 420 and stores the file locally on a Client Database 415. For uploads, the Web-based API 420 is used to transfer the file from the client to the server, along with any instructions or other data required to facilitate storage at the server.

Continuing with reference to FIG. 4, the server application executes multiple Windows Services 430, 445 for downloading and uploading data. Thus, the Web API 420 formats an upload from the client into a Server Folder 440. In this example, the folder is represented as JSON data in a text file; however, other data formats generally known in the art may be used similarly. The Server Upload Window Service(s) 445 receives the Server Folder 440 from the Web API 420 and enters the Server Folder 440 into one or more Remote Databases 425. If a request is received to download data, the Download Window Service(s) 430 retrieves the requested data from the Remote Databases 425. The data is then formatted as a Client Download Folder 435 which is downloaded to the Local Database 415 in the client application via the Web API 420 and the Client Windows Service 410.

As noted above, the Client Application includes a GUI which presents data to the user and allows the user to make modifications to the data which can then be uploaded to the Server Application. In general, data format generally known in the art may be adapted to enhance the presentation of the data and facilitate the user's interaction with the data. For example, in some embodiments, data records are presented in an interactive tabular format referred to herein as a “dynamic worksheet.” Each row of a dynamic worksheet specifies the data corresponding to a particular data record. Users can interact with the record directly to modify information in the data record or to delete the data record altogether. In some embodiments, administrative elements may be provided in the dynamic worksheet to prevent modifications of data records by unauthorized users. For example, in response to receiving a request to modify a particular data record, the system may check the administrative privileges of the currently logged in user to confirm that the user has permission to modify the dynamic worksheet. Following any modification, the user may also be presented with an opportunity to upload the modifications to the server for immediate updating of the server's database. As noted above, the system can operate in an online or offline mode, with scheduled synchronizations between user devices and the server to ensure that the user's data remains current. With the option to immediately synchronize a modification to a data record, the user has the opportunity to ensure that the updates are immediately available at the server rather than waiting for the scheduled synchronization to occur. Thus, updates can be pushed to other users immediately.

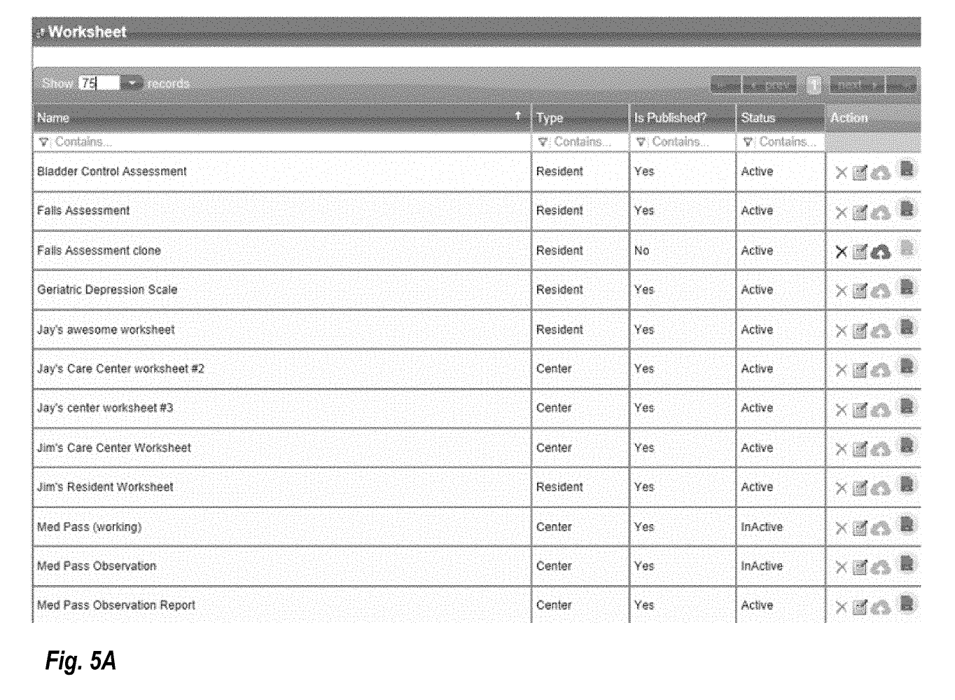

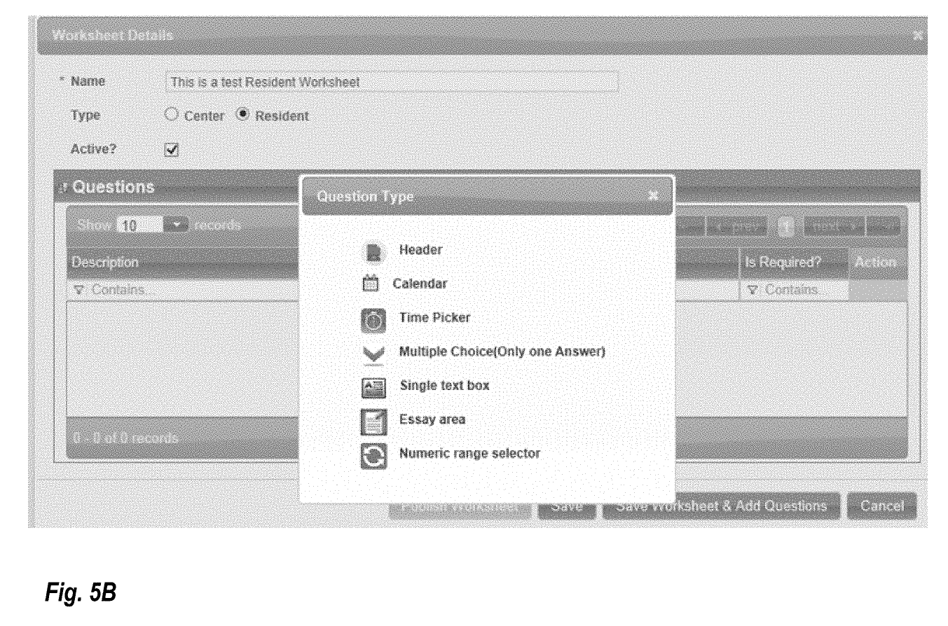

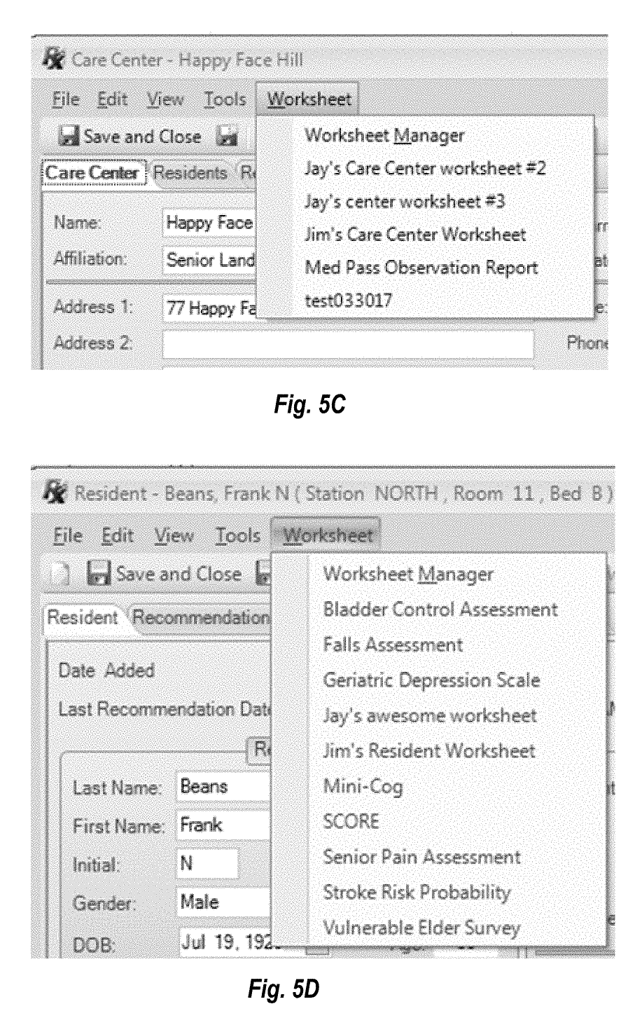

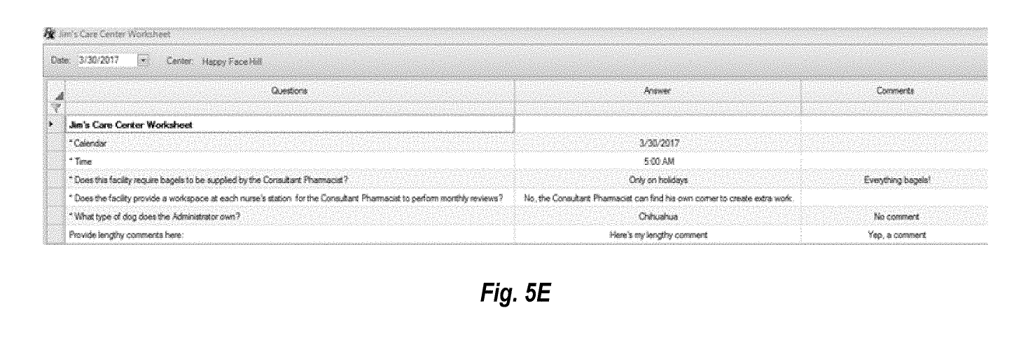

FIGS. 5A-5E provide an example workflow for generating dynamic worksheets according to some embodiments. FIG. 5A shows an example management console which displays the availability of a plurality of dynamic worksheets. For each dynamic worksheet, the console indicates the type of dynamic worksheet, whether it is published, and the current status (i.e., active or inactive). Additionally, an “Action” column provides options for modifying, updating, and deleting individual dynamic worksheets.

A new dynamic worksheet can be created by the organization using several data elements for asking questions, such as illustrated in FIG. 5B. In this example, the organization is presented with seven different options for adding interface elements. The “Header” element provides a field for specifying general information about the worksheet. For example, for a worksheet with resident data, the header may ask for the resident's name and facility. The “Calendar” and “Time Picker” interface elements provide fields for specifying the date and time, respectively, in the spreadsheet. The rest of the interface elements shown in FIG. 5B may be used for structuring specific questions needed to complete the spreadsheet. Each interface element allows the organization to specify a question and the interface for accepting responses. Thus, the “Multiple Choice” element may be used to specify a multiple choice question, while the “Single text box” element may be used to provide a text field for a response. For longer responses, the “Essay area” element may be used to create a text box capable of receiving longer text entry from the user. Finally the “Numeric range selector” interface element allows the organization to specify questions where the response is a numeric range of values.

Once the dynamic worksheet is completed, it can be published and made available to all other users via synchronization with the server. These dynamic worksheets may then be accessed by the user at the facility level or the individual resident level as shown in FIGS. 5C and 5D, respectively. Finally, FIG. 5E provides an example of a test completed dynamic worksheet.

Although the MRR functionality described herein is discussed with reference to a client application executable on a client computing device, it should be noted that the functionality provided by the client application may be delivered in a Software as a Service (SaaS) architecture in other embodiments of the present invention. In this way, the consultant pharmacist can use a web browser or mobile application (app) to access a client application hosted on the server computing device. The servers hosting the application may be configured to support Single Tenancy or Multi-Tenancy within a server infrastructure leveraging Cloud Computing technology. Such an architecture may obviate the need for synchronization of the data, as all data may be stored on the server while providing elasticity of the computer and storage resources. Alternatively, the client application may be desired with functionality that allows it to be executed offline. For example, in one embodiment, the client application is implemented as a progressive web app. As is understood in the art, a progress web app is a hybrid of a web page and a mobile application which uses service workers and local storage to be connectivity independent, thus allowing the client application to be used offline or on low-quality networks.

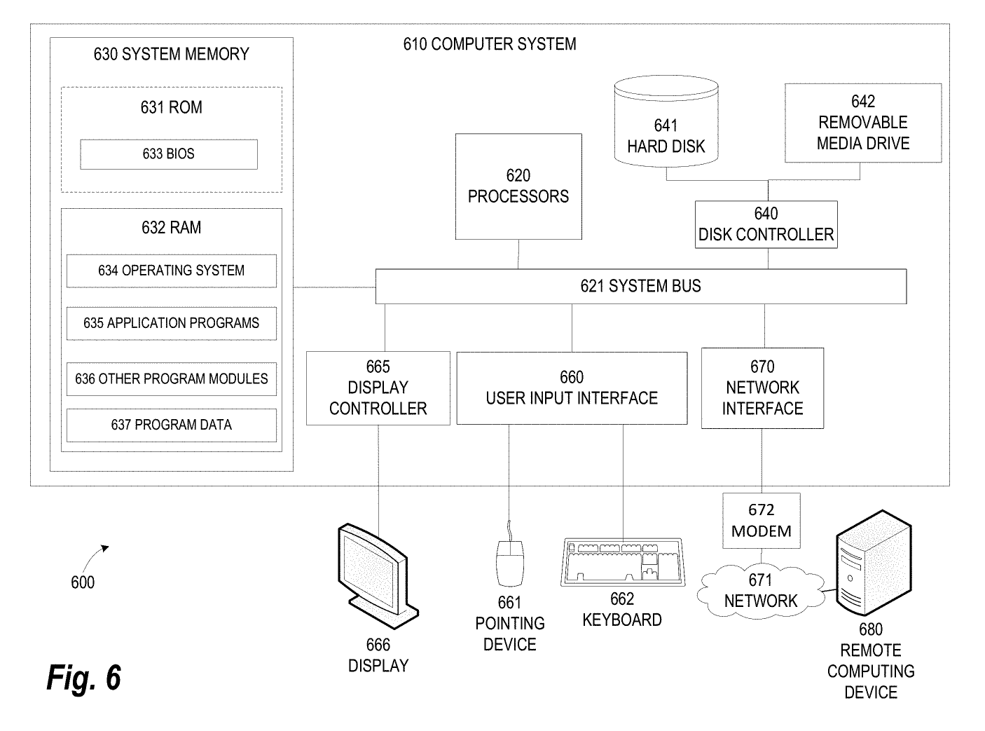

FIG. 6 illustrates an exemplary computing environment 600 within which embodiments of the invention may be implemented. For example, the computing environment 600 may be used to implement the Client Computing Device 117 and/or the Server Computing Device 103 illustrated in the system 100 of FIG. 1. The computing environment 600 may include computer system 610, which is one example of a computing system upon which embodiments of the invention may be implemented. Computers and computing environments, such as computer system 610 and computing environment 600, are known to those of skill in the art and thus are described briefly here.

As shown in FIG. 6, the computer system 610 may include a communication mechanism such as a bus 621 or other communication mechanism for communicating information within the computer system 610. The computer system 610 further includes one or more processors 620 coupled with the bus 621 for processing the information. The processors 620 may include one or more central processing units (CPUs), graphical processing units (GPUs), or any other processor known in the art. In some embodiments, a cloud-based computing environment may be used for implementing the systems and methods described herein. Techniques such as server virtualization can be used to maximize the use of hardware resources.

The computer system 610 also includes a system memory 630 coupled to the bus 621 for storing information and instructions to be executed by processors 620. The system memory 630 may include computer readable storage media in the form of volatile and/or nonvolatile memory, such as read only memory (ROM) 631 and/or random access memory (RAM) 632. The system memory RAM 632 may include other dynamic storage device(s) (e.g., dynamic RAM, static RAM, and synchronous DRAM). The system memory ROM 631 may include other static storage device(s) (e.g., programmable ROM, erasable PROM, and electrically erasable PROM). In addition, the system memory 630 may be used for storing temporary variables or other intermediate information during the execution of instructions by the processors 620. A basic input/output system (BIOS) 633 containing the basic routines that help to transfer information between elements within computer system 610, such as during start-up, may be stored in ROM 631. RAM 632 may contain data and/or program modules that are immediately accessible to and/or presently being operated on by the processors 620. System memory 630 may additionally include, for example, operating system 634, application programs 635, other program modules 636 and program data 637.

The computer system 610 also includes a disk controller 640 coupled to the bus 621 to control one or more storage devices for storing information and instructions, such as a hard disk 641 and a removable media drive 642 (e.g., floppy disk drive, compact disc drive, tape drive, and/or solid state drive). The storage devices may be added to the computer system 610 using an appropriate device interface (e.g., a small computer system interface (SCSI), integrated device electronics (IDE), Universal Serial Bus (USB), or FireWire).

The computer system 610 may also include a display controller 665 coupled to the bus 621 to control a display 666, such as a liquid crystal display (LCD) or light emitting diode (LED) monitor, for displaying information to a computer user. The computer system includes an input interface 660 and one or more input devices, such as a keyboard 662 and a pointing device 661, for interacting with a computer user and providing information to the processors 620. The pointing device 661, for example, may be a mouse, a trackball, or a pointing stick for communicating direction information and command selections to the processors 620 and for controlling cursor movement on the display 666. The display 666 may provide a touch screen interface which allows input to supplement or replace the communication of direction information and command selections by the pointing device 661.

The computer system 610 may perform a portion or all of the processing steps of embodiments of the invention in response to the processors 620 executing one or more sequences of one or more instructions contained in a memory, such as the system memory 630. Such instructions may be read into the system memory 630 from another computer readable medium, such as a hard disk 641 or a removable media drive 642. The hard disk 641 may contain one or more datastores and data files used by embodiments of the present invention. Datastore contents and data files may be encrypted to improve security. The processors 620 may also be employed in a multi-processing arrangement to execute the one or more sequences of instructions contained in system memory 630. In alternative embodiments, hard-wired circuitry may be used in place of or in combination with software instructions. Thus, embodiments are not limited to any specific combination of hardware circuitry and software.

As stated above, the computer system 610 may include at least one computer readable medium or memory for holding instructions programmed according to embodiments of the invention and for containing data structures, tables, records, or other data described herein. The term “computer readable medium” as used herein refers to any medium that participates in providing instructions to the processor 620 for execution. A computer readable medium may take many forms including, but not limited to, non-volatile media, volatile media, and transmission media. Non-limiting examples of non-volatile media include optical disks, solid state drives, magnetic disks, and magneto-optical disks, such as hard disk 641 or removable media drive 642. Non-limiting examples of volatile media include dynamic memory, such as system memory 630. Non-limiting examples of transmission media include coaxial cables, copper wire, and fiber optics, including the wires that make up the bus 621. Transmission media may also take the form of acoustic or light waves, such as those generated during radio wave and infrared data communications.

The computing environment 600 may further include the computer system 610 operating in a networked environment using logical connections to one or more remote computers, such as remote computer 680. Remote computer 680 may be a personal computer (laptop or desktop), a mobile device, a server, a router, a network PC, a peer device or other common network node, and typically includes many or all of the elements described above relative to computer system 610. When used in a networked environment, computer system 610 may include modem 672 for establishing communications over a network 671, such as the Internet. Modem 672 may be connected to bus 621 via user network interface 670, or via another appropriate mechanism.

Network 671 may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between computer system 610 and other computers (e.g., remote computer 680). The network 671 may be wired, wireless or a combination thereof. Wired connections may be implemented using Ethernet, Universal Serial Bus (USB), RJ-11 or any other wired connection generally known in the art. Wireless connections may be implemented using Wi-Fi, WiMAX, and Bluetooth, infrared, cellular networks, satellite or any other wireless connection methodology generally known in the art. Additionally, several networks may work alone or in communication with each other to facilitate communication in the network 671.

The embodiments of the present disclosure may be implemented with any combination of hardware and software. In addition, the embodiments of the present disclosure may be included in an article of manufacture (e.g., one or more computer program products) having, for example, computer-readable, non-transitory media. The media has embodied therein, for instance, computer readable program code for providing and facilitating the mechanisms of the embodiments of the present disclosure. The article of manufacture can be included as part of a computer system or sold separately. For example, in some embodiments, the article of manufacture can be included as part of an Internet of Things (IoT) device such as a smart bed, a smart cart/pill dispenser, or other networked medical equipment.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

A graphical user interface (GUI), as used herein, comprises one or more display images, generated by a display processor and enabling user interaction with a processor or other device and associated data acquisition and processing functions. The GUI also includes an executable procedure or executable application. The executable procedure or executable application conditions the display processor to generate signals representing the GUI display images. These signals are supplied to a display device which displays the image for viewing by the user. The processor, under control of an executable procedure or executable application, manipulates the GUI display images in response to signals received from the input devices. In this way, the user may interact with the display image using the input devices, enabling user interaction with the processor or other device.

The functions and process steps herein may be performed automatically or wholly or partially in response to user command. An activity (including a step) performed automatically is performed in response to one or more executable instructions or device operation without user direct initiation of the activity.

The system and processes of the figures are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. As described herein, the various systems, subsystems, agents, managers and processes can be implemented using hardware components, software components, and/or combinations thereof. No claim element herein is to be construed under the provisions of 35 U.S.C. 112(f), unless the element is expressly recited using the phrase “means for.”

1. A computer-implemented method for performing Medication Regimen Reviews (MRR), the method comprising:
synchronizing a local medication regimen database stored on a client computing device with a remote medication regimen database stored on a server computing device, wherein the local medication regimen database includes medication regimens for patient residing at one or more facilities;
creating a plurality of medication regimen recommendations based on input received via a client interface on the client computing device, wherein each medication regimen recommendation is created by a recommendation process comprising:
receiving a selection of a patient via the client interface,
retrieving a medication regimen corresponding to the patient from the local medication regimen database,
retrieving recommendation phrasing text from a local recommendation phrasings database stored on the client computing device, wherein the recommendation phrasing text is automatically retrieved from the local recommendation phrasings database in response to a macro executed on the client computing device via the client interface;
generating a description of one or more changes to the medication regimen and a recommendation classification describing an MRR activity based on the recommendation phrasing text;
creating the medication regimen recommendation based on the description and the recommendation classification; and
storing the plurality of medication regimen recommendations in a local medication recommendations database on the client computing device.

2. The method of claim 1, wherein synchronization of the local medication regimen database is performed automatically in response to the client computing device receiving an alert message from the server computing device.

3. The method of claim 1, further comprising:
synchronizing a local prescription drug plan (PDP) database stored on the client computing device with a remote PDP database stored on the server computing device, wherein each PDP database comprises a listing of medication coverage for one or more PDPs;
identifying a patient PDP based on the identification of a particular patient;
creating a customized formulary from the local PDP database comprising a listing of medication and coverage information for each medication corresponding to the patient PDP;
in response to receiving user selection of a specific medication in the customized formulary, automatically displaying one or more therapeutic alternatives to the specific medication;
receiving a selection of a specific therapeutic alternative to the specific medication, wherein the description of one or more changes to the medication regimen is generated using the specific therapeutic alternative to the specific medication.

4. The method of claim 1, further comprising:
synchronizing a local clinical monograph database stored on the client computing device with a remote clinical monograph database stored on the server computing device, wherein the local clinical monograph database comprises a plurality of clinical monographs corresponding to medications in the local medication regimen database;
in response to receiving a request to display a clinical monograph corresponding to a specific medication in a particular patient's medication regimen, retrieving a specific clinical monograph from the local clinical monograph database;
displaying the specific clinical monograph in the client interface.

5. The method of claim 1, further comprising:
synchronizing a local medication interactions database stored on the client computing device with a remote medication interactions database stored on the server computing device, wherein the local medication interactions database comprises descriptions of medication interactions corresponding to medications in the local medication regimen database;
retrieving specific medication interactions corresponding to a particular patient's medication regimen from the local medication interaction database;
displaying the specific medication interactions in the client interface.

6. The method of claim 1, further comprising:
receiving an outcome record via input into the client interface, wherein the outcome record comprises a description of an outcome resulting from performance of a specific medication regimen recommendation; and
storing the outcome record on the client computing device in a local outcome record database.

7. The method of claim 6, wherein the outcome record further comprises (i) a description of an outcome resulting from performance of the specific medication regimen recommendation, (ii) a first number specifying a number of adverse drug interactions identified after performing the specific medication regimen recommendation, and (iii) a second number specifying number of adverse drug interactions avoided after performing the specific medication regimen recommendation.

8. The method of claim 6, wherein the outcome record comprises a description of a medication order change which occurred as a result of performance of the specific medication regimen recommendation.

9. The method of claim 8, wherein the description of the medication order change comprises psychoactive medication classification information related to the medication order changes.

10. The method of claim 6, further comprising:
synchronizing the local outcome record database stored on the client computing device with a remote outcome record database stored on the server computing device; and
synchronizing the local medication recommendations database stored on the client computing device with a remote medication recommendations database stored on the server computing device.

11. The method of claim 10, further comprising:
receiving an identification of a facility and a report generation request via the client interface;
identifying a list of patients that are residing in the facility;
retrieving a plurality of medication recommendations corresponding to the list of patients from the local medication recommendations database or the remote medication recommendations database; and
retrieving a plurality of outcome records corresponding to the list of patients from the local medication recommendations database or the remote outcome record database; and
generating a plurality of reports based on the plurality of medication recommendations and the plurality of outcome records.

12. The method of claim 11, further comprising:
tracking time spent by a user in performing the recommendation process,
wherein the plurality of reports includes at least one report indicating the time spent.

13. A computer-implemented method for performing MRRs, the method comprising:
creating a plurality of medication regimen recommendations based on input received via a web-based client interface on a client computing device, wherein each medication regimen recommendation is created by a recommendation process comprising:
receiving a selection of a patient via the web-based client interface,
retrieving a medication regimen corresponding to the patient from a medication regimen database stored on a server computing device,
retrieving recommendation phrasing text from a recommendation phrasings database stored on the server computing device in response to receipt of a phrasing request via the web-based client interface;
generating a description of one or more changes to the medication regimen and a recommendation classification describing an MRR activity based on the recommendation phrasing text; and
creating the medication regimen recommendation based on the description and the recommendation classification; and
storing the plurality of medication regimen recommendations in a medication recommendations database on the server computing device.

14. The method of claim 13, further comprising:
generating a PDP database stored on the server computing device, wherein the PDP database comprises a listing of medication coverage for one or more PDPs;
identifying a patient PDP based on the identification of a particular patient;
creating a customized formulary from the PDP database comprising a listing of medication and coverage information for each medication corresponding to the patient PDP;
in response to receiving user selection of a specific medication in the customized formulary, automatically displaying one or more therapeutic alternatives to the specific medication;
receiving a selection of a specific therapeutic alternative to the specific medication,
wherein the description of one or more changes to the medication regimen is generated using the specific therapeutic alternative to the specific medication.

15. The method of claim 14, further comprising:
in response to receiving a request to display a clinical monograph corresponding to a specific medication in a particular patient's medication regimen, retrieving a specific clinical monograph from a monograph database store on the server computing device;
displaying the specific clinical monograph in the web-based client interface.

16. The method of claim 13, further comprising:
retrieving specific medication interactions corresponding to a particular patient's medication regimen from the a medication interaction database stored on the server computing device;
displaying the specific medication interactions in the web-based client interface.

17. The method of claim 13, further comprising:
receiving an outcome record via input into the web-based client interface, wherein the outcome record comprises a description of an outcome resulting from performance of a specific medication regimen recommendation; and
storing the outcome record on the server computing device in an outcome record database.

18. The method of claim 17, wherein the outcome record further comprises (i) a description of an outcome resulting from performance of the specific medication regimen recommendation, (ii) a first number specifying a number of adverse drug interactions identified after performing the specific medication regimen recommendation, and (iii) a second number specifying number of adverse drug interactions avoided after performing the specific medication regimen recommendation.

19. The method of claim 17, further comprising:
receiving an identification of a facility and a report generation request via the web-based client interface;
identifying a list of patients that are residing in the facility;
retrieving a plurality of medication recommendations corresponding to the list of patients from the medication recommendations database; and
retrieving a plurality of outcome records corresponding to the list of patients from the outcome record database; and
generating a plurality of reports based on the plurality of medication recommendations and the plurality of outcome records.

20. A system for performing MRRs on a client computing device, the system comprising:
a plurality of local databases comprising a medication recommendations database and an outcome record database;
a report upload queue synchronized to an MRR reports database stored on a server computing device; and
a client application configured to (i) generate one or more MRR reports using medication recommendations database and the outcome record database, and (ii) enter each MRR report into the report upload queue.
